(19) United States
(12) Patent Application Publication
(10) Pub. No.: US 2017/0112373 A1
(43) Pub. Date: Apr. 27, 2017

(54) VISUAL ACUITY TESTING METHOD AND PRODUCT

(71) Applicant: Gobiquity, Inc., Scottsdale, AZ (US)

(72) Inventors: Andrew A. Burns, Scottsdale, AZ (US); Darcy Wendel, Palos Verdes Estates, CA (US); Tommy H. Tam, Walnut Creek, CA (US); James M. Foley, Peoria, AZ (US); John Michael Tamkin, Pasadena, CA (US); Peter-Patrick de Guzman, Los Angeles, CA (US)

(21) Appl. No.: 15/333,039

(22) Filed: Oct. 24, 2016

Related U.S. Application Data
(60) Provisional application No. 62/245,811, filed on Oct. 23, 2015.
(60) Provisional application No. 62/245,820, filed on Oct. 23, 2015.

(51) Int. Cl. A61B 3/032; A61B 3/14; A61B 5/00; A61B 3/00

(52) U.S. Cl. CPC A61B 3/032; A61B 3/0025; A61B 3/0033; A61B 3/0058; A61B 3/14; A61B 5/7275; A61B 5/0013; A61B 3/0091; A61B 3/0008; A61B 2562/0219

(57) ABSTRACT

Systems and methods are provided for managing, optimizing subject information, recommending ophthalmologic assessments, and performing diagnostic assessments. The system includes a computing device having an image-capturing device and a display. The system includes a computer application that is executable on the computing device and operable to receive information regarding a subject, recommend ophthalmologic tests based on the information received, and perform ophthalmologic assessments on a subject. Performance of the ophthalmologic assessments causes the application to generate information regarding the ophthalmologic health of the subject, analyze the information generated, and present results of the analysis on the display. The ophthalmologic assessments may include a visual acuity assessment causing the computing device to display a plurality of visual acuity targets, receive user input regarding position of the visual acuity targets, and assess the subject's visual acuity based on the user input received.

### CROSS-REFERENCE TO RELATED APPLICATIONS

This application claims priority to U.S. Provisional Application No. 62/245,811, filed Oct. 23, 2015, entitled “PHOTOREFRACTION METHOD AND PRODUCT;” and claims priority to U.S. Provisional Application No. 62/245,820, filed Oct. 23, 2015, entitled “VISUAL ACUITY TESTING METHOD AND PRODUCT;” which are hereby incorporated by reference in their entirety.

### FIELD OF INVENTION

The present invention is directed to systems and methods for managing ophthalmologic subject information, recommending ophthalmologic assessments, and performing diagnostic assessments. The present invention is further directed to systems and methods for performing, documenting, recording, and analyzing visual acuity assessments.

### BACKGROUND

Managing ophthalmologic patient information is burdensome and requires management of charts and files. It may be difficult to manage large quantities of files and properly identify risk factors associated with each patient. Diagnosing ophthalmologic conditions requires training and a proctor may not be familiar with the symptoms of every ophthalmologic test. Moreover, the proctor must be adequately trained to administer ophthalmologic tests using the equipment provided.

In previously-implemented solutions, a visual acuity wall chart is hung on a wall for administering a visual acuity test. An appropriate distance (e.g., 20 feet) from the wall chart is measured and marked for taking the visual acuity test. In one aspect of the test, a subject or patient is positioned at the distance marked and requested to read lines from the wall chart wherein each line corresponds to a level of visual acuity. However, this method requires a minimum amount of space for the appropriate distance and may not be entirely accurate. A proctor administering the test is required to keep track of every step of the test and determine which line of the wall chart the subject is attempting to read. In some instances, the proctor may intentionally or unintentionally help the subject during the test or inflate the score of the subject. Training may be required for the proctor to properly administer the test.

In another case of testing visual acuity, a visual acuity testing apparatus is typically implemented that projects a chart, such as a Snellen chart, onto a surface positioned at a predetermined distance from the subject. The visual acuity testing apparatus is installed at a position relative to a chair such that the subject will observe letters on the projected chart as if spaced apart from the subject at an appropriate test distance (e.g., 20 feet). Installation of the visual acuity testing apparatus is a custom operation that depends on the dimensions of the environment in which it is located, adding cost and complexity. Additional pieces of equipment (e.g., mirror, control system) may also be required to adequately administer the test and determine visual acuity.

### BRIEF DESCRIPTION OF THE DRAWINGS

FIG. 1 illustrates a block diagram of a system for managing ophthalmologic subject information, recommending ophthalmologic assessments, and performing diagnostic or screening assessments; and for performing, documenting, recording, and analyzing visual acuity assessments.

FIG. 2 illustrates a front view of a computing device in accordance with an embodiment of the present invention.

FIG. 3 illustrates a back view of the computing device of FIG. 2.

FIG. 4 illustrates a schematic view of the computing device of FIG. 2.

FIG. 5 illustrates a homepage of an application in accordance with an embodiment of the present invention.

FIG. 6 illustrates a patient list of the application of FIG. 5.

FIG. 7 illustrates a patient profile screen of the application of FIG. 5.

FIG. 8 illustrates a result screen of the application of FIG. 5.

FIG. 9A illustrates a vision guide tool of the application of FIG. 5.

FIG. 9B illustrates first demographic information entry on the vision guide tool of FIG. 9A.

FIG. 9C illustrates second demographic information entry on the vision guide tool of FIG. 9A.

FIG. 9D illustrates the vision guide tool of FIG. 9A having an expanded test list.

FIG. 10A illustrates a test information section of the application of FIG. 5A.

FIG. 10B illustrates test procedure images of the test information section of FIG. 10A.

FIG. 10C illustrates test procedure description of the test information section of FIG. 10A.

FIG. 10D illustrates exemplary test images of the test information section of FIG. 10A.

FIG. 11A illustrates a diagnosis tool of the application of FIG. 5.

FIG. 11B illustrates an explanation screen of the diagnosis tool of FIG. 11A.

FIG. 12 illustrates a statistics tool of the application of FIG. 5.

FIG. 13 illustrates a process for performing an ophthalmologic assessment using the application of FIG. 5.

FIG. 14A illustrates a first step of a tutorial in the process of FIG. 13.

FIG. 14B illustrates a second step of the tutorial of FIG. 14A.

FIG. 14C illustrates a third step of the tutorial of FIG. 14A.

FIG. 14D illustrates a fourth step in the tutorial FIG. 14A.

FIG. 15A illustrates a first step of a comprehension assessment in the process of FIG. 13.

FIG. 15B illustrates a second step of the comprehension assessment of FIG. 15A.

FIG. 16 illustrates an assessment phase of the process of FIG. 13.

FIG. 17 illustrates instructions for performing an assessment in the assessment phase of FIG. 16.

FIG. 18 illustrates the positioning of an operator and a subject during the visual acuity assessment.

FIG. 19 illustrates a first arrangement of visual acuity targets displayed for conducting a visual acuity assessment at a first level of visual acuity.

FIG. 20 illustrates visual acuity, target information stored on the computing device of FIG. 2.

FIG. 21 illustrates a second arrangement of visual acuity targets displayed for conducting the visual acuity assessment of FIG. 19.

FIG. 22 illustrates visual acuity targets displayed for conducting a visual acuity assessment at a second level of visual acuity.

FIG. 23 illustrates a third arrangement of visual acuity targets in displayed for conducting a visual acuity assessment at a third level of visual acuity.

FIG. 24 illustrates visual acuity targets of a second type for conducting a visual acuity assessment.

FIG. 25 illustrates a results screen indicating that risk factors may be associated with the subject.

### DETAILED DESCRIPTION

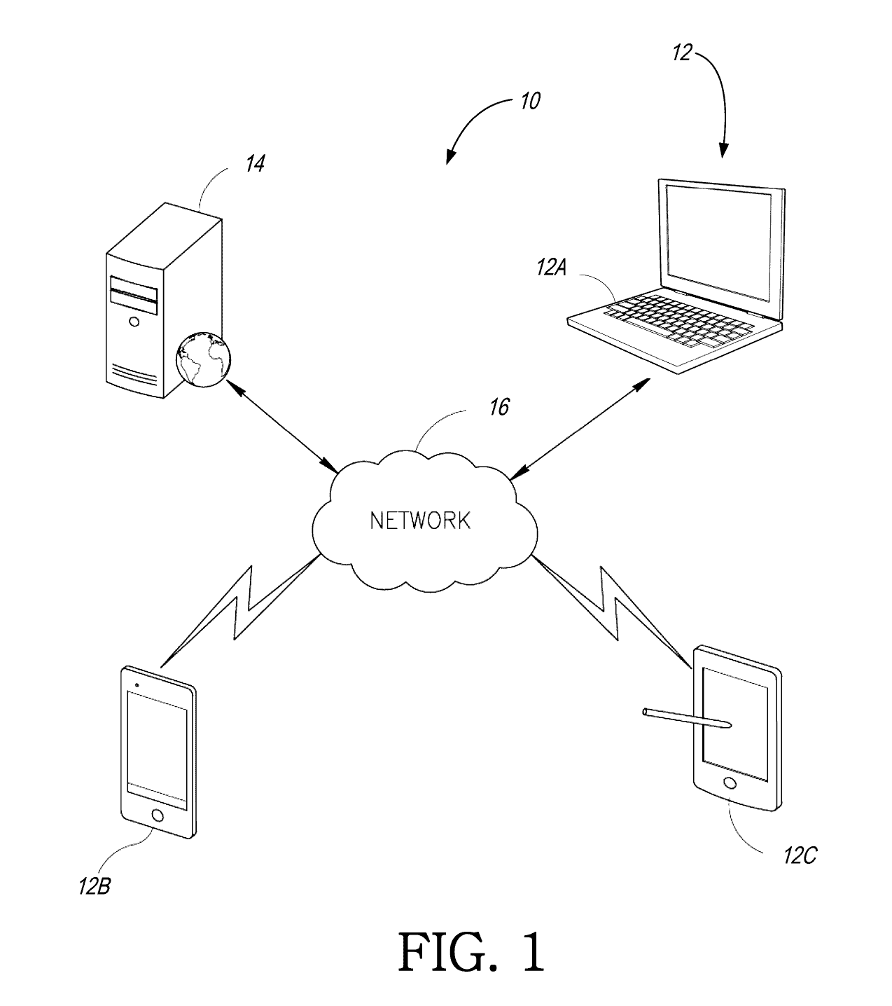

A system and methods for managing ophthalmologic subject information, recommending ophthalmologic assessments, and performing diagnostic or screening assessments is provided according to the present disclosure. FIG. 1 illustrates a simplified version of a system 10 that may be used to provide the functionality described herein. The system 10 includes at least one computing device 12 (e.g., computing devices 12A, 12B, 12C) communicatively coupled to at least one server computing device 14 over a network 16 (e.g., internet, cellular network, ad-hoc network). While the system 10 is illustrated as including the server computing device 14, those of ordinary skill in the art will appreciate that the system 10 may include any number of server computer devices that each perform the functions of the server computing device 14 or cooperate with one another to perform those functions. Further, while the server computing device 14 is illustrated as being connected to the three computing devices 12A-12C, those of ordinary skill in the art appreciate that the server computing device may be connected to any number of computing devices and the server computing device is not limited to use with any particular number of computing devices.

The computing devices 12A-12C are each operated by a user, such as a physician, another healthcare provider, a parent, or the like. The computing devices 12A-12C may each include a conventional operating system configured to execute software applications and/or programs. By way of non-limiting examples, in FIG. 1, the computing device 12A is illustrated as a personal computer (e.g., a laptop), the computing device 12B is illustrated as a smart phone, and the computing device 12C is illustrated as a tablet computer. Generally, the computing devices 12A-12C may include devices that are readily commercially available (e.g., smart phones, tablet computers, etc.), and/or may include devices specifically configured for this particular application. The computing devices 12A-12C may be located remotely from the server computing device 14.

The computing devices 12A-12C each include an image-capturing device (e.g., a camera), and may also include a light-generating device (e.g., a “flash”). A computer application or software may be provided on the computing devices 12A-12C operable to use the image-capturing device and/or the light-generating device to capture images of patients' eyes. In some instances, the light-generating device is located close to the lens of the image-capturing device.

Each of the computing devices 12A-12C also includes a screen display that provides a means to frame the subject and to assure focus of the image-capturing device. The software of the computing devices 12A-12C controls the duration and intensity of the light or flash generated by the light-generating device.

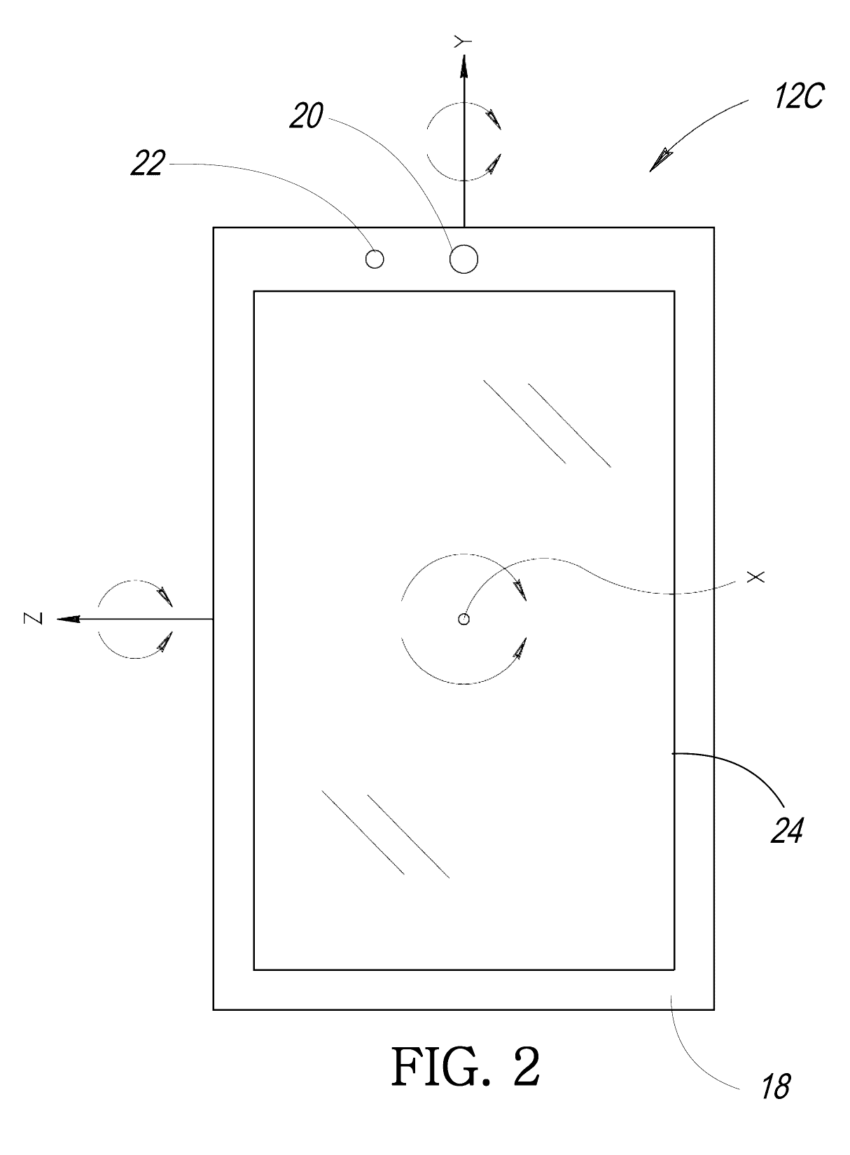

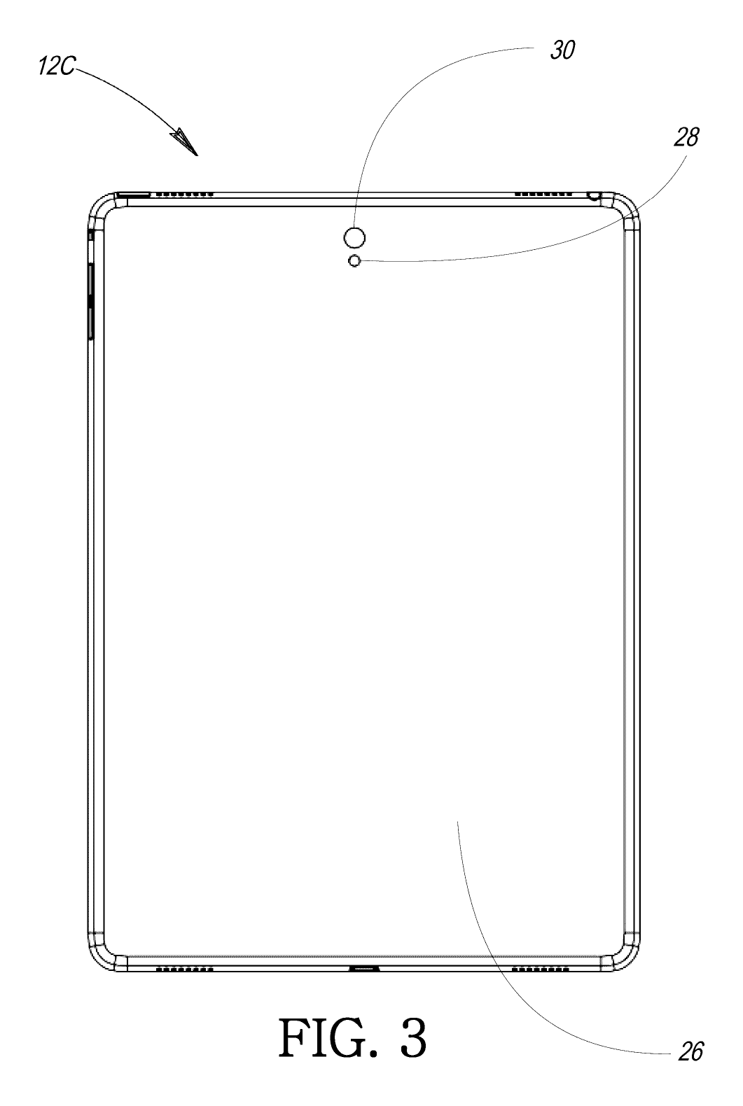

The computing device 12 (e.g. tablet computer 12C) has a front side 18 provided with an image-capturing device 20 (i.e., a camera) and a light-generating device 22 (i.e., a flash), which may be located in close proximity with each other (i.e., separated by a small distance), as shown in FIG. 2. The front side 18 of the computing device 12 also includes a display device 24 for real-time display. The display device 24 may be touch-sensitive (e.g., touchscreen) and operable to control the aspects of the computing device 12, such as the operating system, applications, and hardware (e.g., image-capturing device, light-generating device). A back side 26 of the computing device 12 (e.g., tablet computer 12C) may also be provided with an image-capturing device 28 (i.e., camera) and a light-generating device 30 (i.e., flash) located in close proximity with each other, as shown in FIG. 3. The tablet computer 12C may be iPad produced by Apple®, by way of non-limiting example.

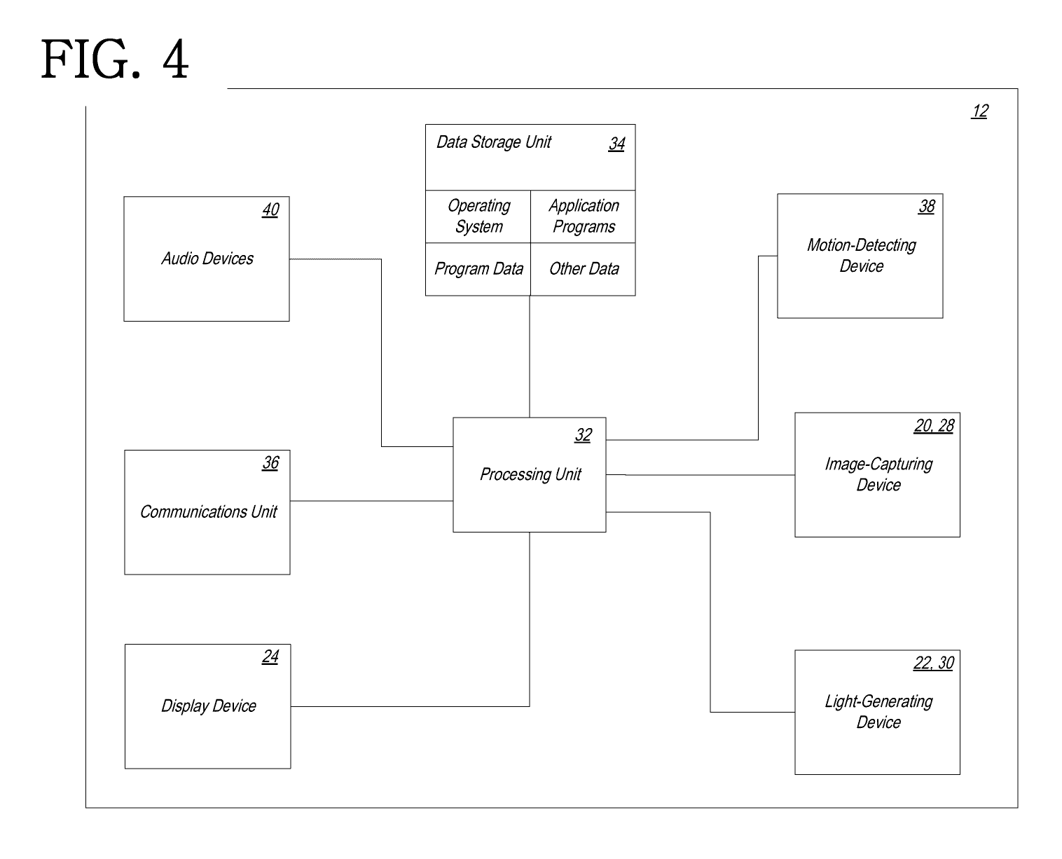

The computing device 12 includes a processing unit 32 electronically coupled to several components, including a data storage unit 34, a communications unit 36, a motion-detecting unit 38, audio devices 40, the display device 24, the light-generating devices 22 and 30, and the image-capturing devices 20 and 28, as shown in FIG. 4. The processing unit 32 may communicate with and/or control the components by sending and receiving electronic signals, including data and control signals. The data storage unit 34 is a non-transitory storage medium, such as hard drive for reading and writing data thereon, and may include one or more types of memory types (e.g., RAM, ROM, cache) known in the art. The data storage unit 34 may store different types of data, such as an operating system, one or more application programs, program data, and other data (e.g., word documents, media files, etc.). The data storage unit 34 has executable instructions that, as a result of execution on the processing unit 32, cause the processing unit to communicate with and control the components of the computing device.

The processing unit 32 electronically communicates with and controls the other components according to programming data on the data storage unit 34. For example, the processing unit communicates with display device 24 to display images thereon, and receives data from the touch screen of the display device for interacting with the computing device 12. The processing unit 32 sends independent control signals to the image-capturing devices 20 and 28 controlling the settings thereof and causing each of them to capture image data for transmission back to the processing unit 32. The processing unit 32 sends control signals independently to each of the light-generating devices 22 and/or 30 for generating light according to the control signal (e.g., at a specific timing, at a specific brightness, etc.). The processing unit 32 may send and receive data through the communications unit 36, which may be a wireless transceiver (e.g., Bluetooth®, Wi-Fi, cellular). The processing unit 32 may send and receive audio signals to and from the audio devices 40, which may comprise one or more speakers and/or one or more microphones.

The motion-detecting unit 38 is configured to detect movement and/or orientation of the computing device 12 about one or more axes X, Y, and Z, as shown in FIG. 2. The motion-detecting unit 38 may comprise one or more accelerometers electronically coupled to the processing unit 32. The motion-detecting unit 38 produces acceleration data about the one or more axes X, Y, and Z, and outputs the data to the processing unit 32. In some embodiments, the motion-detecting unit 38 may process the acceleration data produced into other forms, such as orientation data. The processing unit 32 receives and processes data from the motion-detecting unit 38, and may perform control functions according to the data received, as described below.

Embodiments of the systems and methods described herein enable conducting ophthalmologic assessments, managing practice and patient information, and sharing assessment results using the capabilities of the computing device 12. Embodiments of the systems and methods include a software program or application 50 executing on the computing device 12. A user may store the application 50 on the data storage unit 34 and activate the application 50 via the display device 24. After initial activation of the application 50, the user may be required to register an account by entering certain information, such as name, profession, practice name, address, phone number, email, etc. The account registered may be associated with the user on the server computing device 14, such that at least some information may be exchanged between the computing device 12 and the server computing device 14 using the application 50. The server computing device 14 may persistently store at least some of the information generated using the application 50 in association with the account. The server computing device 14 provides a remote server that can store practice information, patient information, and test results in a centralized location. The application 50 uses computing device 12 features such as an on-screen keyboard or dialog boxes to enter information. A web application may also be provided that can be accessed by the web browser on any computer, and which may be used to access and manage patient information and test results.

After successful login and registration, the user is directed to a homepage 51 of the application, shown in FIG. 5. From the homepage 51, the user may choose to perform any of the following actions: view information about subjects whose demographic information has previously been entered and not yet been assessed or screened; view information about subjects who have been assessed or screened and their respective test outcome/results (e.g., a screening report); add new subjects to be tested/screened; search for a particular patient/subject; view messages, securely or not, that have been sent by the practice/practitioner through the secure portal; view images of screened patients/subjects with elevated risk factors (e.g., “Great Catches”); ability to share images, assessment details, and results with other practitioners, parents, and application users through a social media interface unique to the application 50; view screening/testing trends including data about their own practice, data about the universe of screeners/users and data population demographics; and access the vision guide.

The user may register a subject or patient to be tested/screened with the application 50, and enter demographic information about the registered subject using input features of the client device, such as a keyboard or a wheel. Alternatively, subject demographic information may be entered via a web application. Users also have the ability to upload certain file types directly into the application 50 that include information about the subject, such as name, date of birth, gender, ethnicity and other demographic information. Optional patient entry may be included in the application 50 in association with other electronic platforms such as electronic medical records with custom API interfaces. All information entered via either method is accessible using cloud-based platform for data storage and retrieval on the server computing device 14, for example.

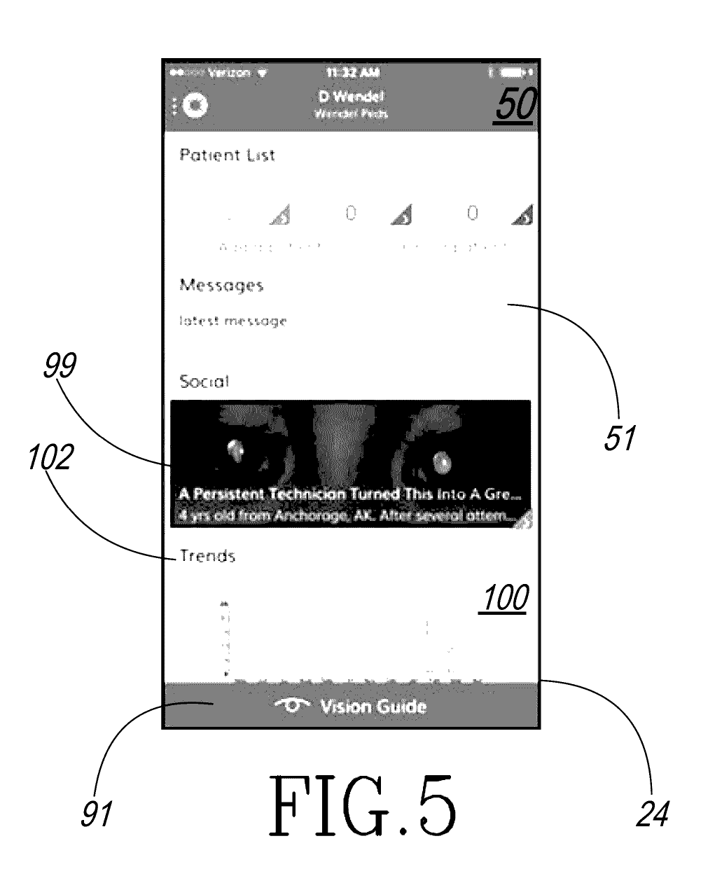

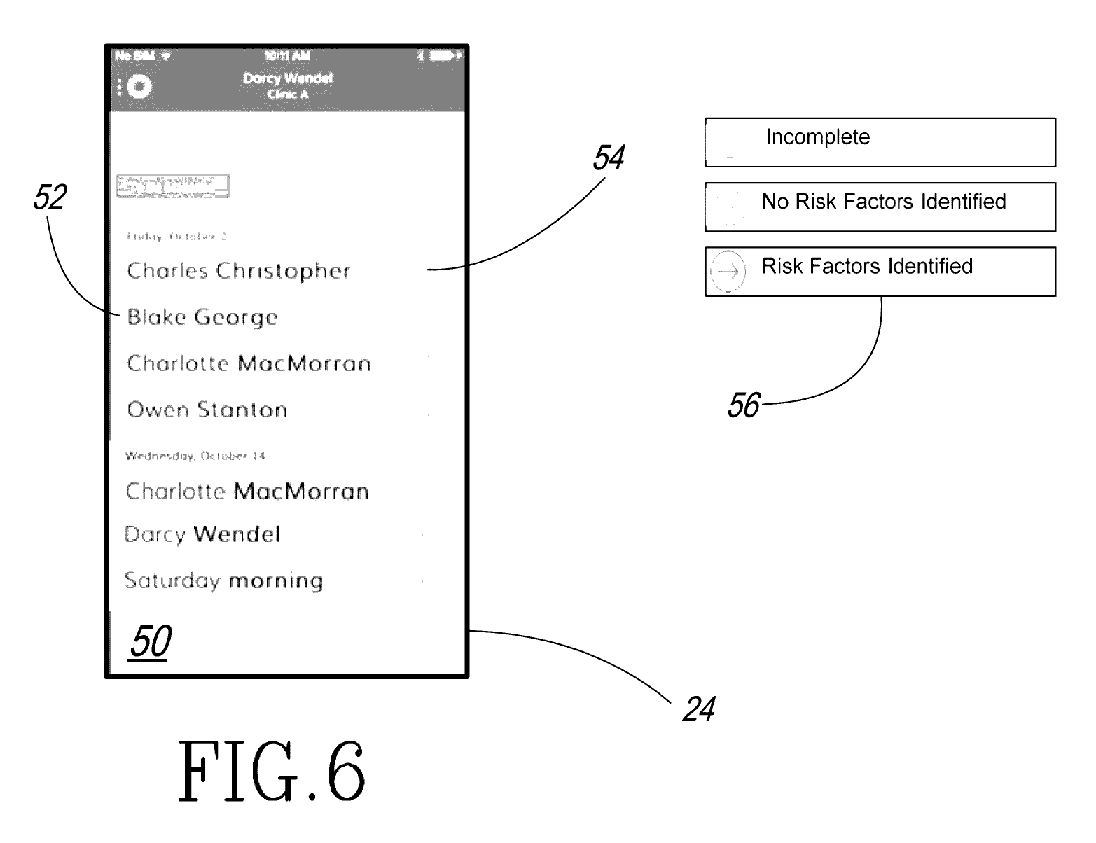

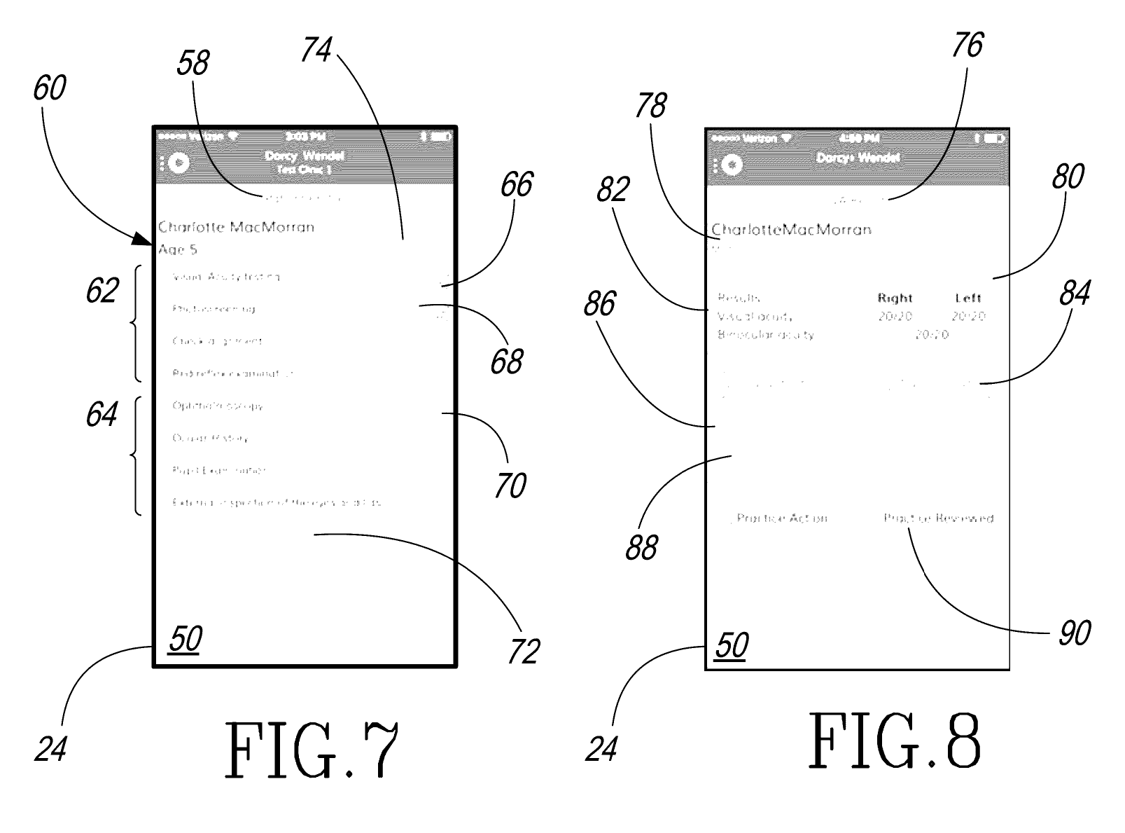

After registering the patient or subject using the application 50, the patient will be displayed on the display device 24 and become available for selection in a patient list 52 section of the application 50, as shown in FIG. 6. Patient information entered in the application 50 will be stored in a storage device (e.g., the data storage unit 34, the server computing device 14) in association with the patient's account. The patient list 52 displays those subjects who have been entered and not screened, as well as subjects screened within a specified timeframe (e.g., the past 7 days). The patient list 52 may display demographic information (e.g., name, date of birth) and the status of one or more assessments. The application 50 may provide other aspects at the patient list 52, such as the capability for the user to search, add and update patient information, or to look up and run reports on patient test results. A patient status icon 54 may be displayed for easy identification of current status in the list, the patient status icon being selected from one or more of a plurality of status types 56, such as “incomplete”, “to be screened”, “no risk factors”, and “risk factors.” The patient list 52 may be sorted by status type 56 to enable the user to quickly identify and sort patients based on risk or screening status. Tapping on any patient with an “incomplete” status type 56 will move to a patient profile screen 58, shown in FIG. 7. Any subject selected with a result will move to a results screen 76, as shown in FIG. 8.

After selecting a subject from the patient list 52, the user is directed to the patient profile screen 58 (FIG. 7). The patient profile screen 58 contains the demographic information 60, as well as provides a recommended tests list 62, based on the age, and potentially other demographic information, entered regarding this particular subject. The recommended test list 62 is based on currently published professional organization (e.g., American Academy of Pediatrics, etc.) guidelines for practice. The recommended test list 62 may also be referred to as the “Vision Guide”, described below. The recommended test list 62 displays a predetermined number of the most important tests (e.g., four), in order of priority for age, with the option to expand the list to include other recommended tests 64. Tests associated with an icon 66 of a first color, such as a green icon, or that have a predetermined icon 68 associated therewith, such as a camera, indicate availability to perform the test with the computing device 12. Icons 70 of a second color, such as gray, indicate additional information and instructions for performing a test not available for performance with the computing device 12, as well as future additions to the application 50. A selection 72 (e.g., button) may be provided to add other tests to the list or browse from a list of tests not currently displayed. A selection 74 may be available on the patient profile screen 58 to display more information regarding a subject, such as demographic information. An option may be included to edit the subject's information from the patient profile screen 58. The user has the option to return to the homepage 51 from the patient profile screen 58 or select any of the available assessment by tapping one of the icons. For example, tapping the “Visual Acuity testing” icon will initiate the visual acuity test, described below in greater detail. Those of ordinary skill in the art will appreciate that other ophthalmology tests not illustrated may be provided in the application 50, such as photorefraction.

The application 50 generates information during each assessment regarding the subject. An analysis may be performed using the information generated, from which results may be determined regarding the ophthalmologic health of the subject. Results from previously performed assessments may be calculated and displayed on the results screen 76, such as the Visual Acuity Test Result Screen shown in FIG. 8. The result screen 76 may display patient information 78, such as demographic information or patient identification information. An indication of risk factors 80 may be displayed on the result screen 76 based on information generated during an assessment, the patient's demographic information, and/or the analysis of the assessment. The relevant risk factors may be based on age-specific referral criteria as defined by the American Academy of Pediatrics (see American Academy of Pediatrics Section on Ophthalmology, Committee on Practice and Ambulatory Medicine, American Academy of Ophthalmology, American Association for Pediatric Ophthalmology and Strabismus, and American Association of Certified Orthoptists. Instrument-Based Pediatric Vision Screening Policy Statement. Pediatrics. 2012; 130:983-986), and the American Association of Pediatric Ophthalmology and Strabismus (See Donahue S P, Arthur B, Neely D E, Arnold R W, Silbert D, Rubin J R. Guidelines for automated preschool vision screening: A 10-year, evidence-based update. J AAPOS. 2013; 17(1):4-8).

A results section 82 displaying results particular to the assessment performed or analysis thereof is displayed on the results screen 76. For example, in the Visual Acuity Results screen shown in FIG. 8, the results section 82 displays the calculated visual acuity of each eye (near or distance, or the critical line acuity) and the calculated binocular visual acuity, which are generated as a result of the information generated during the visual acuity assessment. User demographic information (e.g., age, race, and sex) may be used to determine a test threshold that the patient needs to achieve to pass the assessment as the eye changes over time and different criteria are used to determine pass/fail rates based on population demographics. A remote server, such as the server computing device 14 may store test results in a centralized location and all results may be accessible via the application or on the Internet via the remote server. A web application may be accessible via a web application accessible on any web browser or any computer to access and manage test results and patient information.

The result screen 76 may include a test selection 84 (e.g., button, dialog box) for accessing or re-performing the assessment for which the results are displayed or perform other tests. A practice notes section 86 on the result screen 76 provides the ability to enter notes about the subject or the test performed. Information from the assessment, the assessment results, and/or practice notes are accessible on the computing device 12 through the application 50 or through the Internet, and are stored in compliance with HIPAA standards in the cloud, such as on the server computing device 14. A communications tool 88 allows a user to communicate with a third party regarding the test results, such as communications with a vision care professional through the application 50 (i.e., “ask the expert”) or submitting a positive diagnosis of an ophthalmologic condition along with information obtained during the assessment. Practice management tools 90 may be available for tracking actions on assessment results or submitting assessment information or results to a practice for further review.

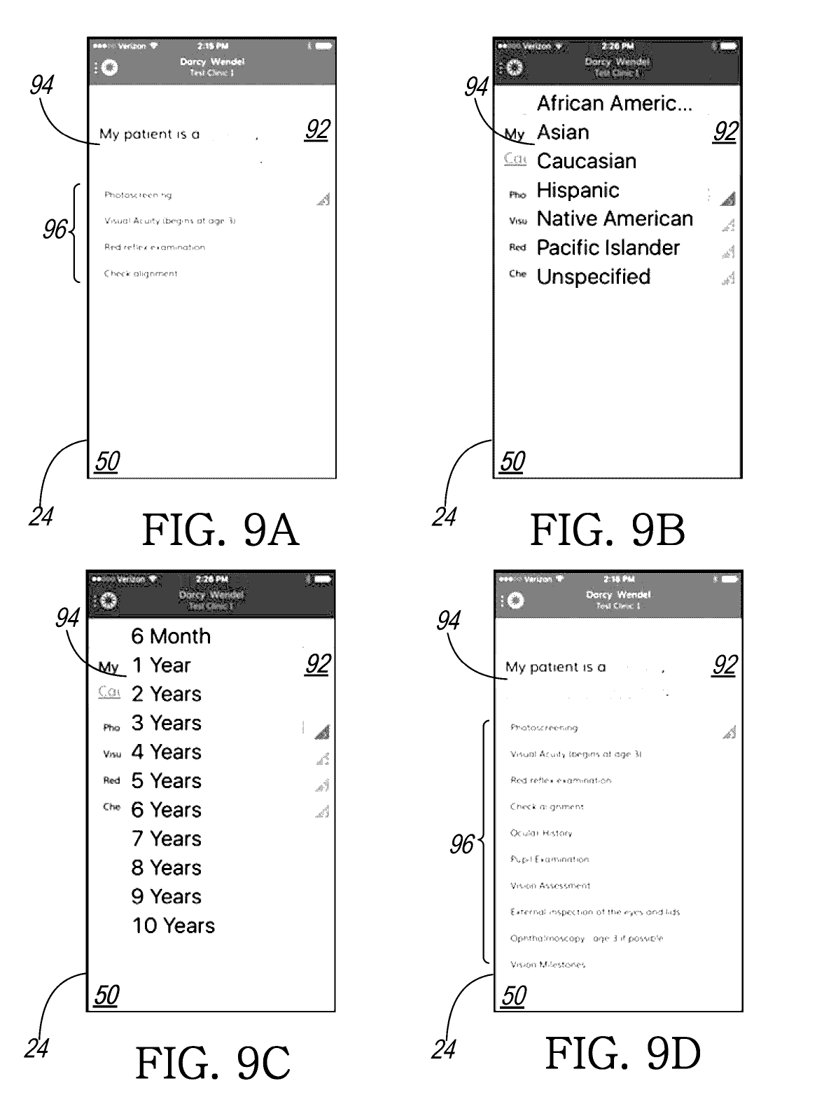

Selecting “Vision Guide” 91 from the homepage 51 brings up the vision guide tool 92 of the application 50, shown in FIGS. 9A through 9D. In the vision guide tool 92, the user can enter demographic information 94 using natural language, via drop down lists or scroll wheels for example, as shown in FIGS. 9B, and 9C, and receive vision screening guidelines based on professional organization recommendations. A top recommended test list 96 for the particular demographic information 94 entered is displayed in the vision guide tool 92, as shown in FIG. 9A. The user has the ability to expand the test list 96 to view all recommended tests, as shown in FIG. 9D. Assessments associated with icons having a first color (e.g., green) represent assessments that can be performed using the application 50, and assessments associated with icons having a second color (e.g., gray) represent tests having additional information (or assessments available in the future through the application 50) for display. Selecting an assessment from the test list 96 having the first color causes the application 50 to initiate the corresponding test, such as the Visual Acuity test described below.

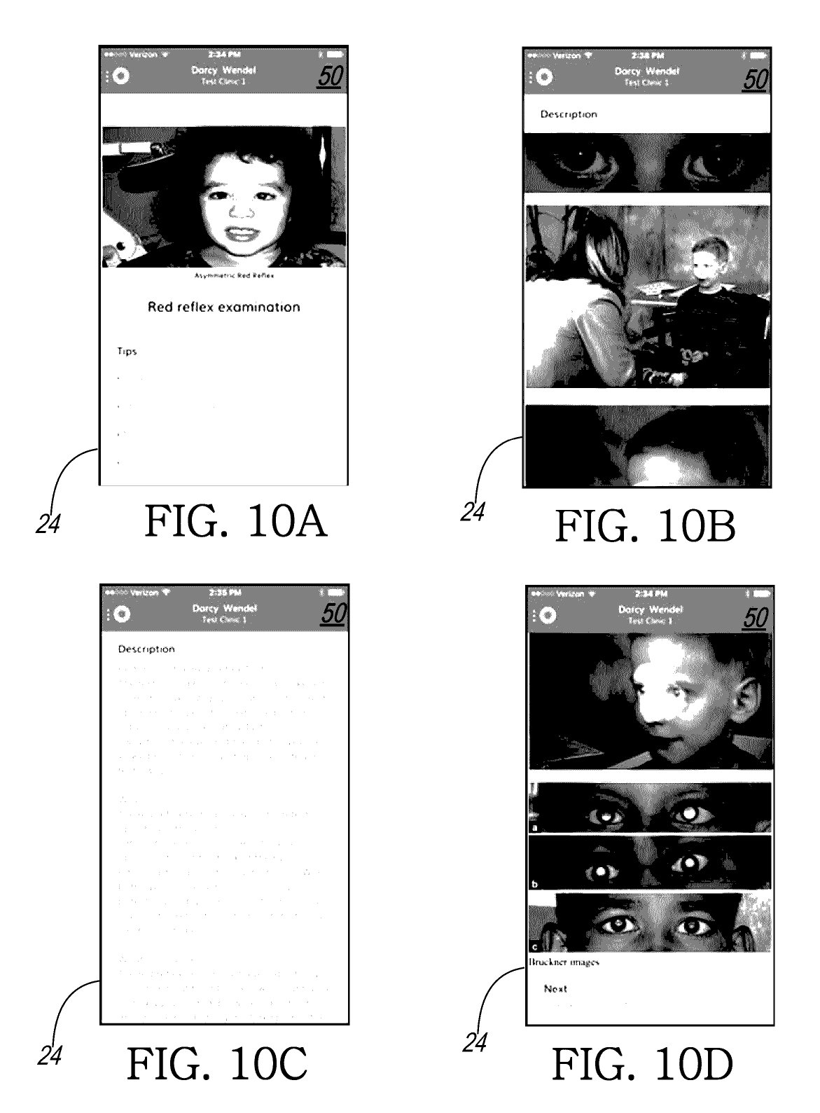

Selecting an assessment from the test list 96 having the second color causes the application 50 to display additional information regarding the corresponding test, as shown in FIGS. 10A through 10D. For instance, if the user selects the “red reflex examination” test illustrated in FIG. 10A, which has an icon having the second color, information about the test is displayed on the display device 24 through the application 50. The additional information may include parameters for performing the test (e.g., environmental conditions, appropriate distance for performing test), and step-by-step instructions for performing the test including text and/or images. The additional information may identify ophthalmologic conditions that may be indicated or detected using the test, and identify characteristics or symptoms that suggest the existence or absence of the ophthalmologic condition.

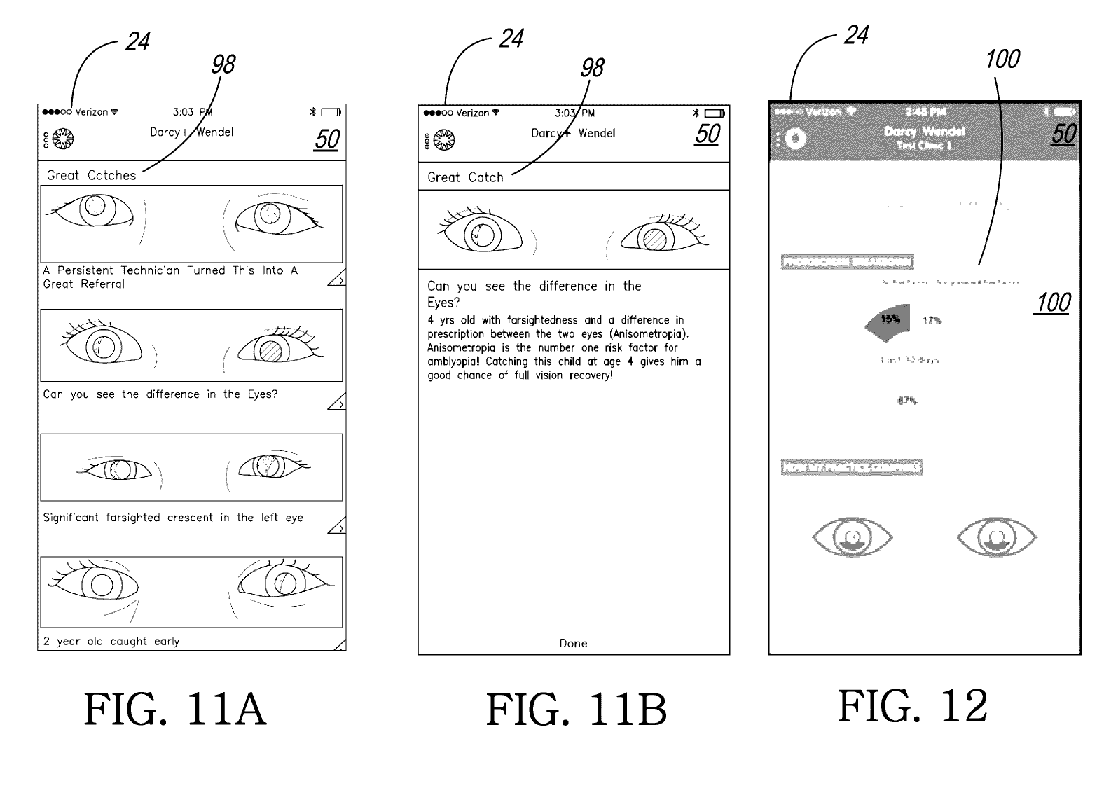

The application 50 includes a risk factor assessment tool 98 that displays images of patients with risk factors on the display device 24, along with a brief description of what has been detected using the application 50, as shown in FIGS. 5, 11A, and 11B. The risk factor assessment tool 98 provides the application 50 with the ability to integrate into a vision assessment (e.g., patient results) that can be shared with eye care professionals or other third parties electronically. The risk factor assessment tool 98 (entitled “Great Catches” in FIGS. 11A and 11B) represents subjects having vision disorders identified by or using the application 50. If the user selects an image in the risk factor assessment tool 98, they will be directed to a list of “great catches,” as shown in FIG. 11A. From the list they can select an image and receive detailed information about that image, as shown in FIG. 11B. The application 50 may include the ability to share images and details through social media channels. The risk factor assessment tool 98 may be accessible via a “Social” selection option 99 on the homepage 51, as shown in FIG. 5.

The application 50 may include a statistics tool for providing statistical analysis regarding ophthalmologic and vision screening, as shown in FIG. 12. The statistics tool may use data collected and stored at the server computing device 14 for performing statistical analyses, and display results 100 of the analyses on the display device 24, as shown in FIGS. 5 and 12. The analyses may be performed using, by way of non-limiting example, age groups, geographic locations, and demographic information. The results may be displayed on a “trends” section 102 on the homepage 51 or on a separate screen (see FIG. 12), and may include number of subjects screened, outcome of screening by risk or type, subject demographic data and may be displayed by user, practice or universe. Selecting the “trends” section 102 from the homepage 51 will direct user to additional trends that may be sorted or filtered by date, demographic data, user, practice or universe.

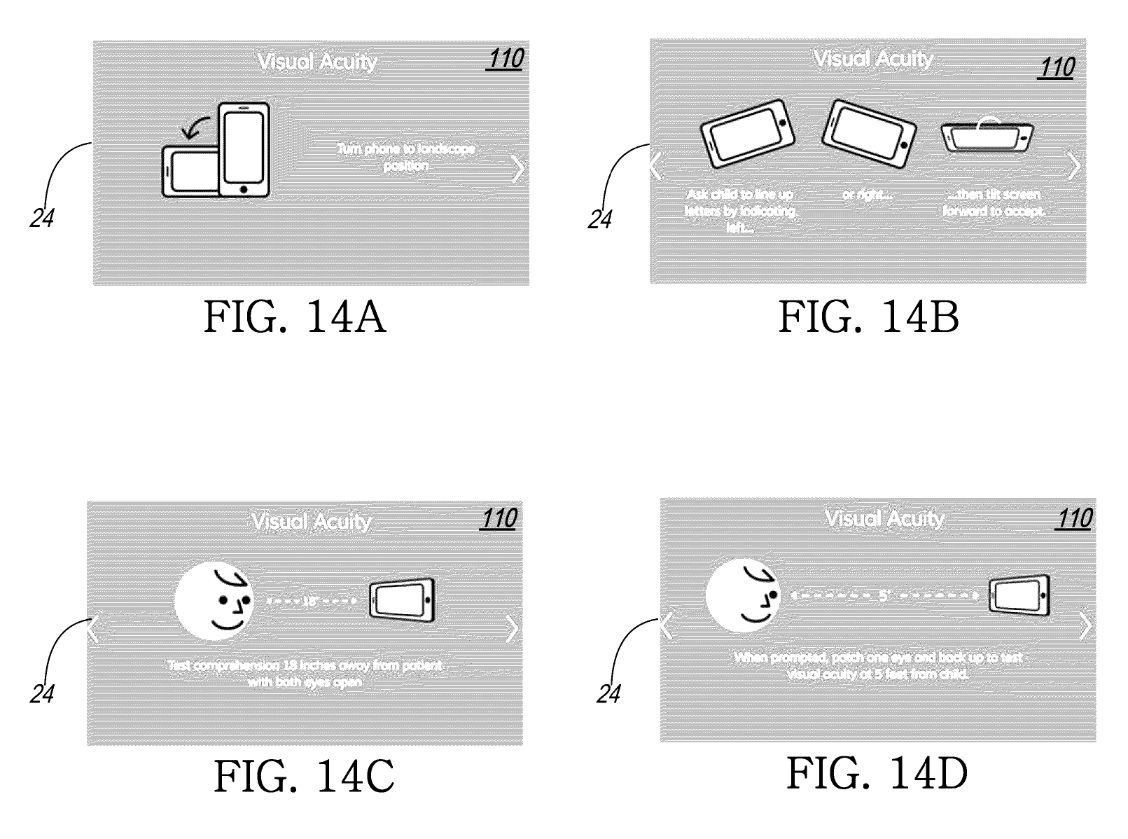

A visual acuity test 110 is part of an integrated suite of mobile vision diagnostics available in the application 50, which includes other diagnostic tests and may include a variety of educational features, as shown in FIGS. 14A through 14D, 15A and 15B, 17, 18, 19, 21, and 22. Visual acuity may be determined using several acceptable methods such as “top down” or threshold acuity, critical line visual acuity, that begins testing a child at the line of visual acuity required for a particular age, or near visual acuity, which tests the ability to see near objects. The visual acuity test 110 is provided to determine the visual acuity (i.e., clarity or sharpness of vision) of a subject using the features of the computing device 12. Test distance may be determined by the age of the individual being tested. Test distances may vary from 1 to 20 feet depending on the type of test and the age of the subject. A proctor or user administers the visual acuity test 110 by holding the display device 24 of the computing device 12 facing the subject. The visual acuity test 110 of the application 50 displays images for the subject on the display device 24, and the subject communicates with the user based on the images displayed. The proctor moves the computing device 12 according to the communications from the subject and guidance of the application 50 to conduct the test. The application 50 processes the movements of the computing device 12 to generate information regarding the visual acuity of the subject.

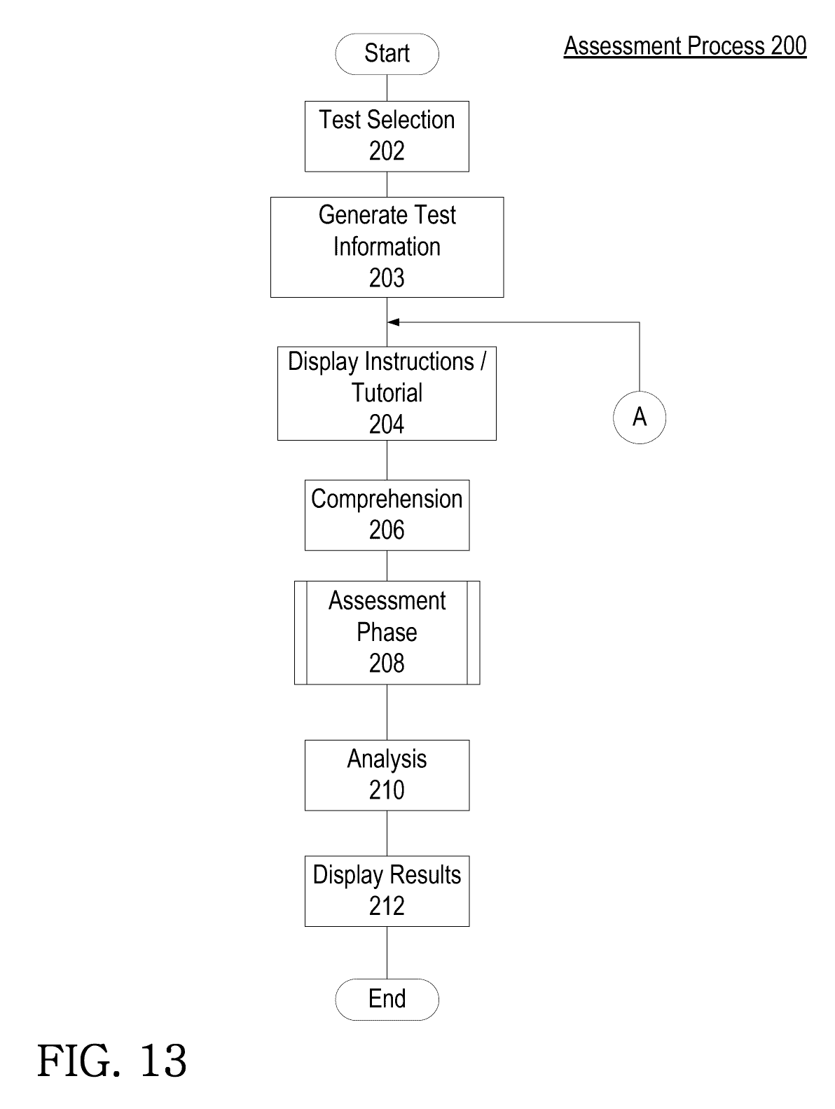

An assessment process 200 for performing the visual acuity test is shown in FIG. 13. In step 202 of the assessment process 200, the computing device 12 receives a selection of one or more assessments to be performed such as the visual acuity test 110, and may additionally receive a sub-selection for the assessment to be performed such as threshold vision acuity, near vision acuity, critical line testing, and/or binocular vision acuity. In step 203, the computing device 12 generates test information for performing the selected assessments. If a visual acuity assessment is selected, for instance, the test information generated in step 203 may specify conditions or criteria for sufficiently evaluating a level of visual acuity. The test information may include information regarding the number of rounds to perform for each level of visual acuity, the levels of visual acuity to be tested, the type of visual acuity targets to be used (e.g., optotypes). The test information may be generated based on settings for the visual acuity test 110 or demographic information 60 of the subject, such as age or race.

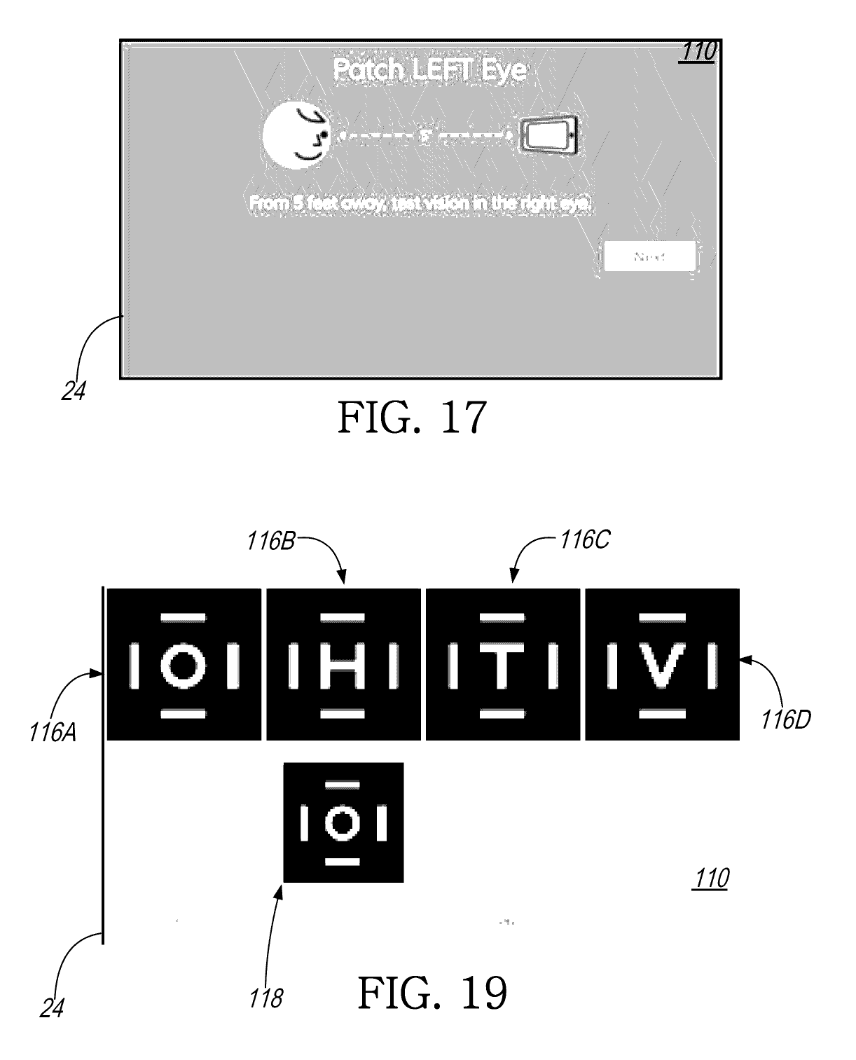

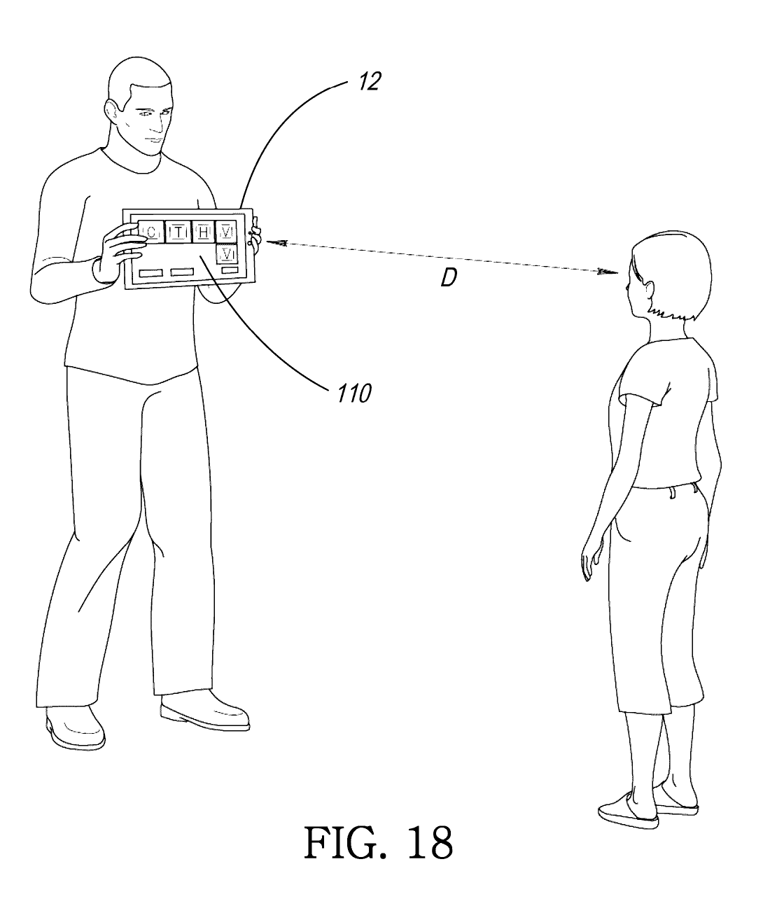

In step 204, the computing device 12 may display instructions 114 on the display device 24 instructing the subject and/or the proctor on as performing the assessment, such as instructing the subject to cover one of their left eye and right eye to ensure no peeking or cheating with the covered eye, as shown in FIG. 17. The computing device 12 may execute or display a tutorial instructing the user on how to perform the assessment selected. The instructions 114 may include audio instructions issued from the speaker of the audio devices 40. The instructions 114 may instruct the proctor and/or the subject to be spaced apart at a proper test distance D for the type of test selected (typically 1-20 feet), which is either measured manually by the proctor or calculated by the mobile device, as shown in FIG. 18. The proctor is required to hold the computing device 12 with the display device 24 facing the subject.

In one embodiment, the application 50 may detect the distance D between the image-capturing device 20 or 28 and the subject, and provide a message or other feedback (e.g., vibration, sound) to the proctor indicating that the test distance is not correct, or that the test distance has changed. The distance D may be measured from the image-capturing device 20 or 28 to the eyes of the subject (i.e., based on interpupilary distance) or an ancillary tool having a known size, such as a sticker or a coin positioned on a face of the subject. The appropriate distance D for performing the visual acuity test may be dependent upon the type of visual acuity test and/or demographic information of the subject (e.g., age, sex, ethnicity). For instance, the distance D is shorter in testing near vision than distance vision. Measurement of the distance D is described in the aforementioned U.S. Provisional Application No. 62/245,811, filed Oct. 23, 2015, entitled “PHOTOREFRACTION METHOD AND PRODUCT;” and U.S. Provisional Patent Application No. 62/245,820, filed on Oct. 23, 2015, entitled “VISUAL ACUITY TESTING METHOD AND PRODUCT,” which are incorporated by reference in their entirety.

As the subject progresses through the test, the application 50 may cause the computing device to present engaging sounds and/or visuals (e.g., graphics and animations) to encourage the subject to pay attention and continue through the test. The application 50 may cause the computing device 12 to generate successful sound and graphics even when the patient fails a step in order to encourage the patient to go on.

In step 206, the computing device 12 may execute a comprehension process to ensure that the subject understands his responsibilities for participating in the selected assessment. In step 208, the computing device 12 performs the selected assessment and generates information regarding performance of the subject during the assessment. The computing device 12 analyzes the information generated during performance of the assessment in step 210, and displays the results of the assessment on the display device 24 based on the analysis. Further description of each step of the assessment process 200 is described in greater detail below.

Upon activating the visual acuity test 110, which may be accessed at the patient profile screen 58 (FIG. 7), the computing device 12 may receive (in step 202) a user selection of one of several different types of visual acuity tests including distance vision, near vision and/or binocular vision. Once the type of visual acuity test 110 is selected by tapping on the visual acuity icon on the display device 24, the application 50 will bring the user to a visual acuity test 110 tutorial (in step 204) that is operable to guide the proctor through the visual acuity screening process by test type, step by step, as shown in FIGS. 14A through 14D. The application 50 includes an option to turn the tutorial feature off once the user becomes familiar with the use of the visual acuity test. In one step of the tutorial process, the visual acuity test 110 indicates an orientation of the computing device that should be maintained during the test, shown in FIG. 14A. One or more screens of the tutorial process indicate actions that the proctor may perform to conduct the visual acuity test 110, and provide instructions for informing the subject on how to interact with the proctor and/or the computing device 12 during the test, as shown in FIG. 14B. One or more of the tutorial screens may provide an indication of the proper distance between the subject and the display device 24 of the computing device 12 based on one or more factors, such as the type of acuity test selected, the test phase, and/or the dimensions of the display device 24 (e.g., 5 feet for the distance vision test). For instance, the tutorial process may cause the display device 24 to display a screen indicating a first distance (e.g., 18 inches) between the display device 24 and the subject during one phase of the visual acuity test 110, as shown in FIG. 14C, and a second distance (e.g., 5 feet) between the subject and the display device 24 during another phase of the test, as shown in FIG. 14D. The first and second distances may correspond to distances for performing different phases of the visual acuity test 110, such as a comprehension phase or an assessment phase, described below. The proper distance between the display device 24 and the subject during the assessment phase of a distance test may be different for different acuity assessment types. For example, the proper distance between the subject and the display device 24 during a near vision assessment may be less than the proper distance during a distance assessment (e.g., 18 inches).

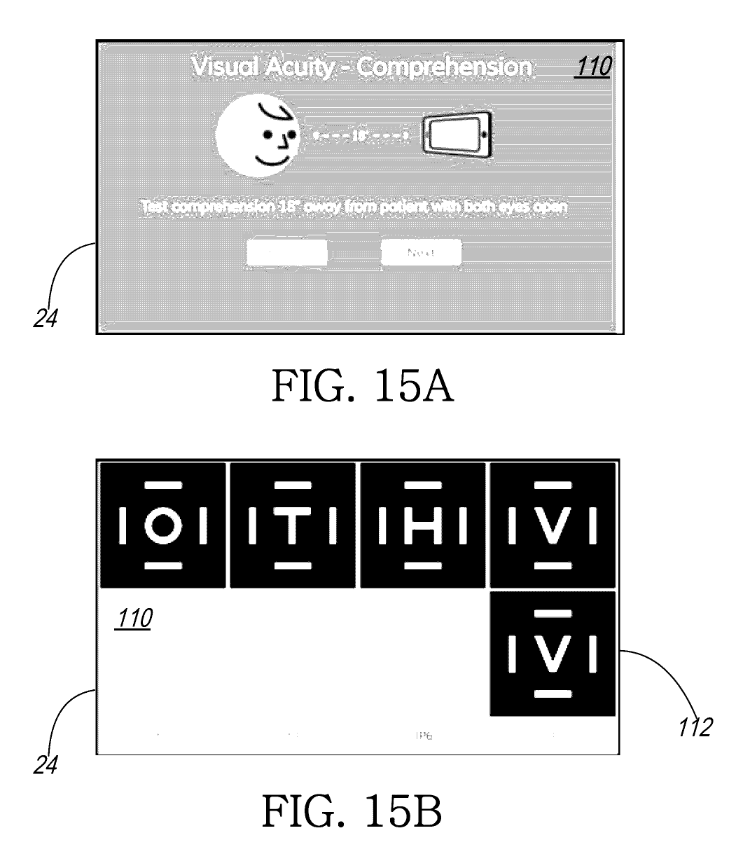

After completing the tutorial in step 204, the user may advance to the comprehension step 206 of the assessment process 200, in which the subject is tested to determine whether the subject understands how to take the test. To test comprehension, the computing device 12 may cause the display device 24 to display instructions to the user to position the display device 24 at a prescribed distance from the subject, as shown in FIG. 15A. Then, the computing device 12 may execute an exemplary step in the assessment phase 208 that the subject should be able to easily pass. the computing device 12 may display large visual acuity targets 112 having a size between 20/63 to 20/200 that the subject should be able to resolve, and the subject is tested (as described below) to ascertain the subject's comprehension of the assessment, or ability to understand how to take the test. If a subject fails comprehension, the test is repeated one or more times. If a subject fails comprehension a predetermined number of times, a message is displayed indicating that the subject failed comprehension. If the subject passes comprehension, the process advances to the next step of the assessment.

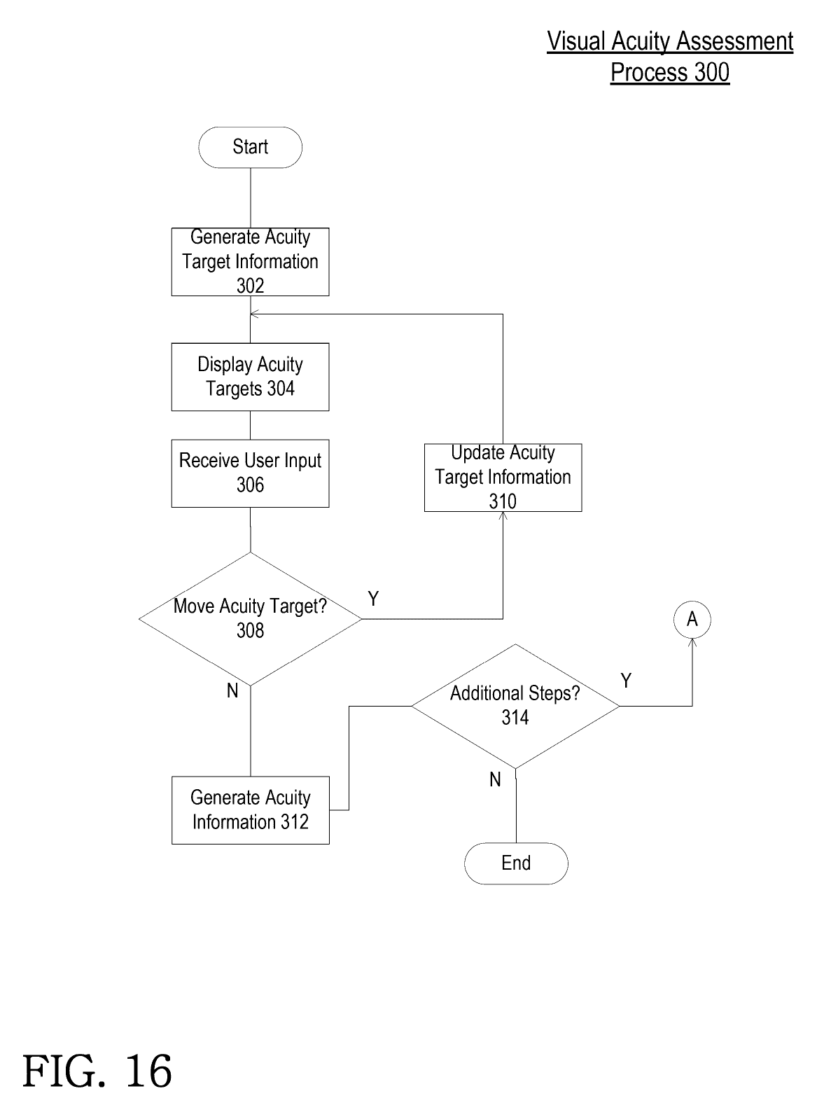

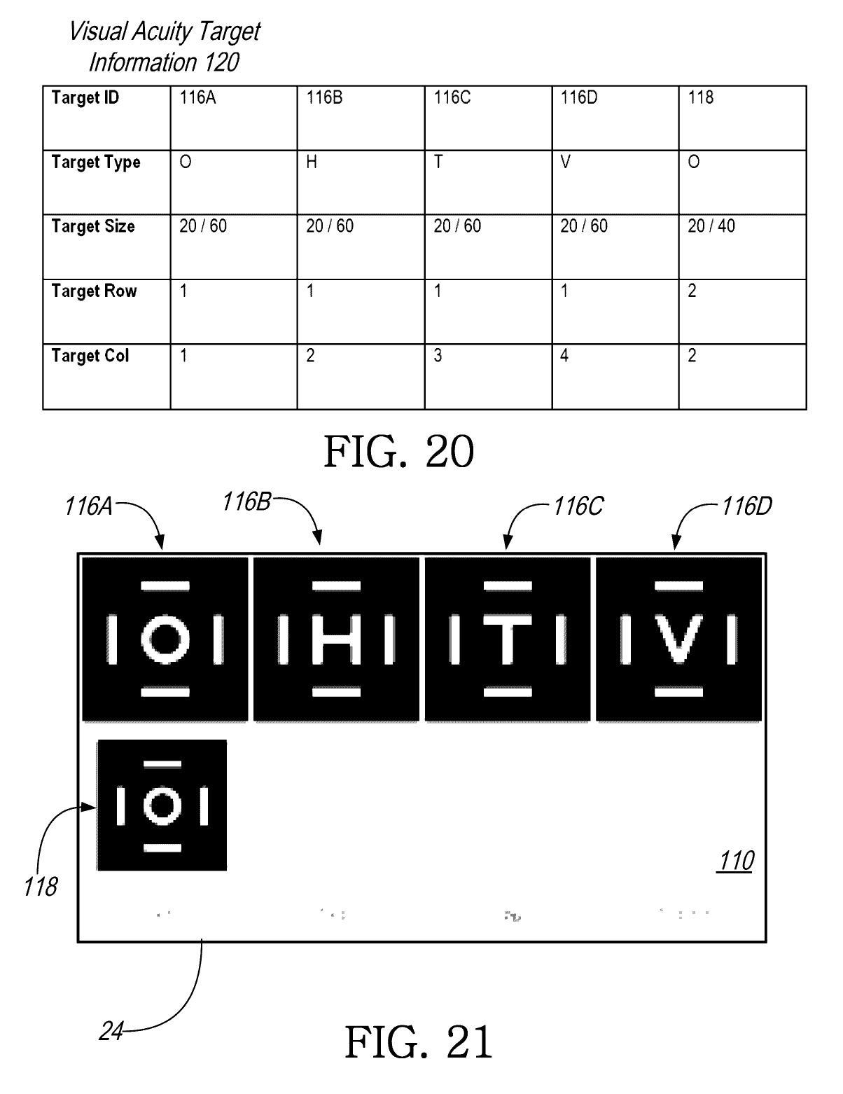

In step 208, the assessment phase of the assessment process 200 is conducted. In the current embodiment, the assessment phase 208 is a visual acuity assessment process 300 (shown in FIG. 16) performed to assess the visual acuity; however, other tests may be performed to assess other ophthalmologic aspects, such as photoscreening for refractive risk factors for amblyopia, for example. The visual acuity assessment process 300 of the visual acuity assessment 110, which begins by generating information regarding the visual acuity targets to be displayed in step 302. The visual acuity test utilizes age specific visual acuity targets or optotypes for a prescribed age range beginning at 3 years of age through adulthood, as shown in FIG. 19. The computing device 12 determines a type of the visual acuity targets to display (e.g., HOW, RKZS, ETDRS or SLOAN), size and position information of first visual acuity targets 116, and size and position information of a second visual acuity target 118. The type, size and position of each visual acuity target may be stored on the data storage unit 34 as visual acuity target information 120, as shown in FIG. 20.

The first visual acuity targets 116 are a plurality of targets each having a same size according to the size information and arranged in a specific arrangement. Each of the first visual acuity targets 116 are different optotypes from one another and are each assigned their own position information. Referring to FIG. 19 for example, the first visual acuity targets 116A-116D are arranged in a line along a horizontal direction respectively in the order O-H-T-V above the second visual acuity target 118. The position information of the first visual acuity targets 116A-116D may correspond to an absolute position in the order (i.e., 116A is in column 1, 116D is in column 4), or may correspond to a position of the first visual acuity targets 116A-116D on the display device 24. The second visual acuity 118 has the same optotype as one of the first visual acuity targets 116 (i.e., 0 in FIG. 19), and is positioned below and adjacent to the first visual acuity targets 116. The position information of the second visual acuity target 118 may be a position of the second visual acuity target relative to the first visual acuity targets (i.e., 118 is at column 2 in FIG. 19), or may be determined as a position of the second visual acuity target 118 on the display device 24. The size information of the second visual acuity target 118 corresponds to the level of visual acuity being tested (e.g., the 20/40 optotype is larger than the 20/20 optotype). The size of the second visual acuity target 118 (corresponding to the size information) is typically smaller than the first visual acuity targets 116, although it may be the same size as the first visual acuity targets 116 in early rounds of testing or depending on the information of the subject (e.g., if the subject is known to have poor visual acuity). The computing device 12 stores the visual acuity target information generated of the first visual acuity targets 116 and the second visual acuity target 118 in the data storage unit 34.

In step 304, computing device 12 displays the first visual acuity targets 116 and the second visual acuity target 118 according to the size and position information stored. The second visual acuity target 118 may be moved relative to the first visual acuity targets 116 according to user input received, as described below. The object of each round of the test is matching the optotype of the second visual acuity target 118 with a corresponding optotype of the plurality of first visual acuity targets 116. For example, in FIG. 19, the object is to move the second visual acuity target 118 having an optotype “0” to a corresponding position adjacent to or directly below the first visual acuity target 116A also having an optotype “0”. The application 50 may cause a speaker of the audio devices 40 to play sounds and/or music accompanying the visual acuity targets to keep the subject interested.

After displaying the visual acuity targets in step 304, the computing device 12 waits to receive user input. The subject is required to indicate an action to take, by communicating with the proctor or issuing a voice command to the computing device 12. For instance, the subject may request to move the second visual acuity target 118 in a particular direction, or select a current position of the second visual acuity target 118 as an accepted answer.

In step 306, computing device 12 receives user input of a predetermined form to perform an action. In the present embodiment, the computing device 12 receives user input via the motion-detecting unit 38, which is configured to output one or more signals indicating a direction and a magnitude of motion detected. The subject may communicate with the proctor to indicate a direction in which the second visual acuity target 118 should move. In response, the proctor should rotate or tilt the computing device 12 about an x-axis direction orthogonal to the surface of the display device 24 (see FIG. 2). The application 50 may move the second visual acuity target 118 in the direction of the rotation or tilt. If the subject indicates that the second visual acuity target 118 should move left on the display device 24, for example, the proctor should rotate the computing device 12 in a counterclockwise direction about the x-axis to move the visual acuity target 118 left on the display. In response to detecting, via a signal received from the motion-detecting unit 38, that the motion of the computing device 12 exceeds a predetermined threshold in a direction, the application 50 may determine that a corresponding user input has been entered. For example, if the proctor rotates the computing device 12 more than 30° in the counterclockwise direction, the application 50 may determine that a user input has been entered to move the second visual acuity target 118 to the left based on the signal received from the motion-detecting unit 38. Once the subject is satisfied that the second visual acuity target 118 matches the first visual acuity target 116 directly above it, the subject may notify the user to accept the current position of the second visual acuity target 118 as an answer or a selected position. In response, the proctor may rotate or tilt the computing device 12 in about the y-axis (see FIG. 2) beyond a predetermined threshold. For instance, the proctor may rotate the computing device 12 more than 30° in a forward direction (i.e., counterclockwise about the y-axis) causing the application to determine that a user input has been entered to accept the current position of the second visual acuity target 118 is a “match” to the first visual acuity target directly above it. The application 50 may generate input information corresponding to the movement of the computing device 12 received.

In some embodiments, the computing device 12 may receive voice commands through a microphone of the audio devices 40 instead of or in addition to the motion detecting device 38. For instance, the application 50 may recognize voice cues or commands, such as “move left” and “move right” instead of movement of the computing device 12, to move the second visual acuity target 118 on the display device 24. The application 50 may recognize a voice command, such as “accept”, to accept the current position of the second visual acuity target 118 as a match to the first visual acuity target 116 directly above it.

After the application 50 determines that a user input has been received in step 306, the assessment process advances to step 308. In step 308, the application determines whether the received user input is a request to move the second visual acuity target 118. If the application 50 determines that the user input received is a request to move the second visual acuity target 118 on the display device 24, the assessment process advances to step 310 to change the position of the second visual acuity target according to the input received. If the application determines that the user input received is not a request to move the second visual acuity target 118, the assessment process advances to step 312.

In step 310, the application 50 updates the position information of the second visual acuity to target 118 according to the user input received in step 306. If the user input received in step 306 is an instruction to move the second visual acuity target 118 to the left on the display device 24, the application 50 may update the target information to update the position information of the second visual acuity target 118 from column 2 (i.e., below the first visual acuity target 1166) to column 1 (i.e., below the first visual acuity target 116A). The assessment process then proceeds back to step 304 at which the acuity targets are displayed on the display device 24 of the computing device 12 according to the updated target information, as shown in FIG. 21.

In step 312, the application 50 generates visual acuity information based on a determination regarding proximity of the second visual acuity target 118 relative to a position of the one of the plurality of first visual acuity targets 116 having the same optotype as the second visual acuity target 118. The application 50 may compare the position information of the second visual acuity target 118 with the position information of the first visual acuity targets 116 to reach the aforementioned determination regarding proximity. If an aspect of the position information of the second visual acuity target 118 matches an aspect of the position information of the first visual acuity target 116 having the same optotype, the application 50 may generate information indicating a positive correlation between the subject's visual acuity and the level of visual acuity being tested. That is, the application 50 may determine that the visual acuity of the subject is sufficient to resolve the second visual acuity target 118 displayed corresponding to the level of visual acuity being tested responsive to a determination that the subject correctly matched the second visual acuity target 118 with the first visual acuity target 116 having the same object type. The application 50 may increase a score for the visual acuity level being tested, for example, if the horizontal position information (i.e., column) of the second visual acuity target 118 is the same as or matches the horizontal position information of one of the first visual acuity targets 116 having the same target type, or maintain or decrease the score otherwise. Alternatively, the application 50 may determine that a selected position of the second visual acuity target 118 is correct it is nearer to the one of the first visual acuity targets 116 having the same target type than the other first visual acuity targets 116. The visual acuity information generated may further be based on a length of time that it takes for the subject to answer. The visual acuity information may be an indicator of one or more risk factors associated with the subject.

In step 314, the application 50 conducts a determination of whether additional steps should be conducted. The application may determine that additional rounds of the visual acuity assessment 110 should be conducted based on the test information generated in step 203 (see FIG. 13), the visual acuity target information generated in step 302, or the visual acuity information generated in step 312. If the requisite number of rounds for the level of visual acuity have been tested or the visual acuity level has otherwise been sufficiently determined, the application 50 may determine that the size of the visual acuity target 118 should be adjusted to test another round or level of visual acuity, and return back to step 204 to conduct another round of the visual acuity assessment. Alternatively, the application 50 may determine to conduct another round of the visual acuity assessment in which the current size of the visual acuity target should be maintained. Other conditions may cause the application 50 to return to step 204 to conduct another round of tests, including a determination that the other one of the left eye and right eye should be tested, a determination that the near vision of the subject should be tested, a determination that the critical line visual acuity should be tested, or a determination that binocular vision of the subject should be tested. Returning to step 204, the application 50 may cause the computing device 12 to display instructions on the display device 24 regarding the next round of testing, such as instructing the subject to cover the other eye or conducting the tutorial. Additionally, the application 50 may cause the computing device to test comprehension of the subject if a different test is performed, such as critical line testing. If the application 50 determines that the aspects of the visual acuity assessment 110 specified in the test information are satisfied, the assessment process may advance to step 210 (see FIG. 13) to perform analysis on the visual acuity information generated in step 312.

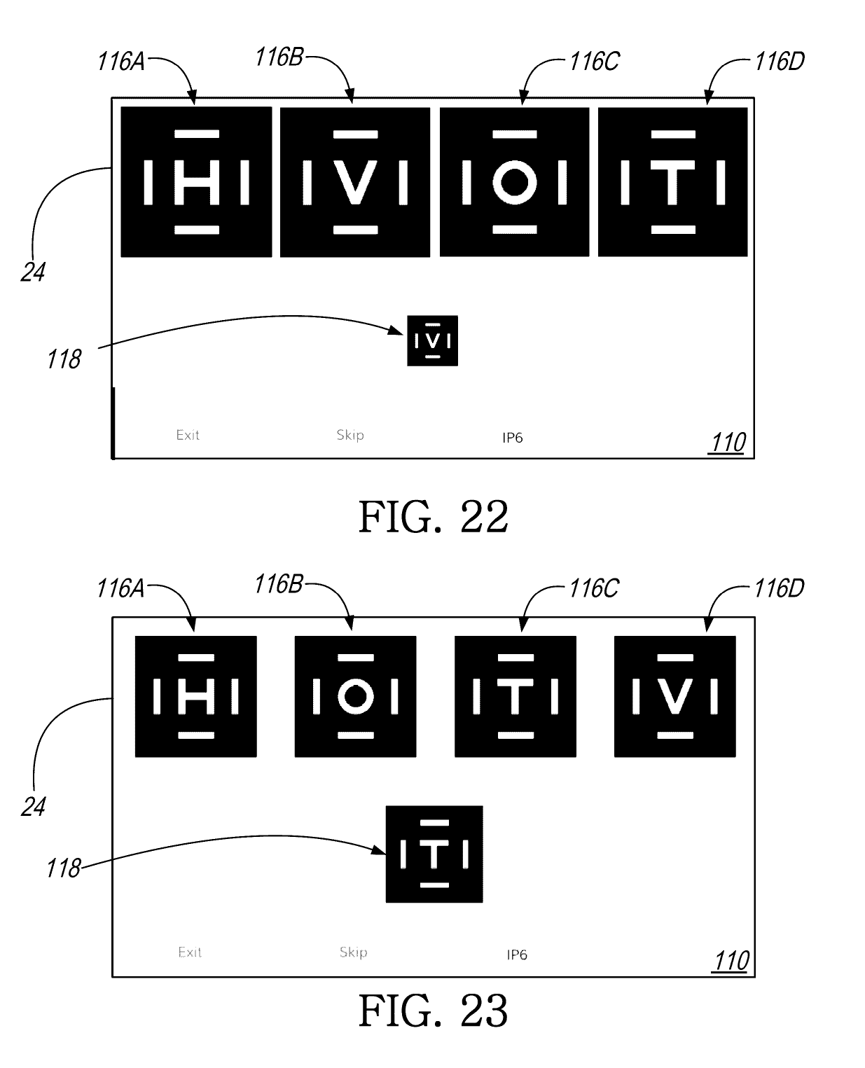

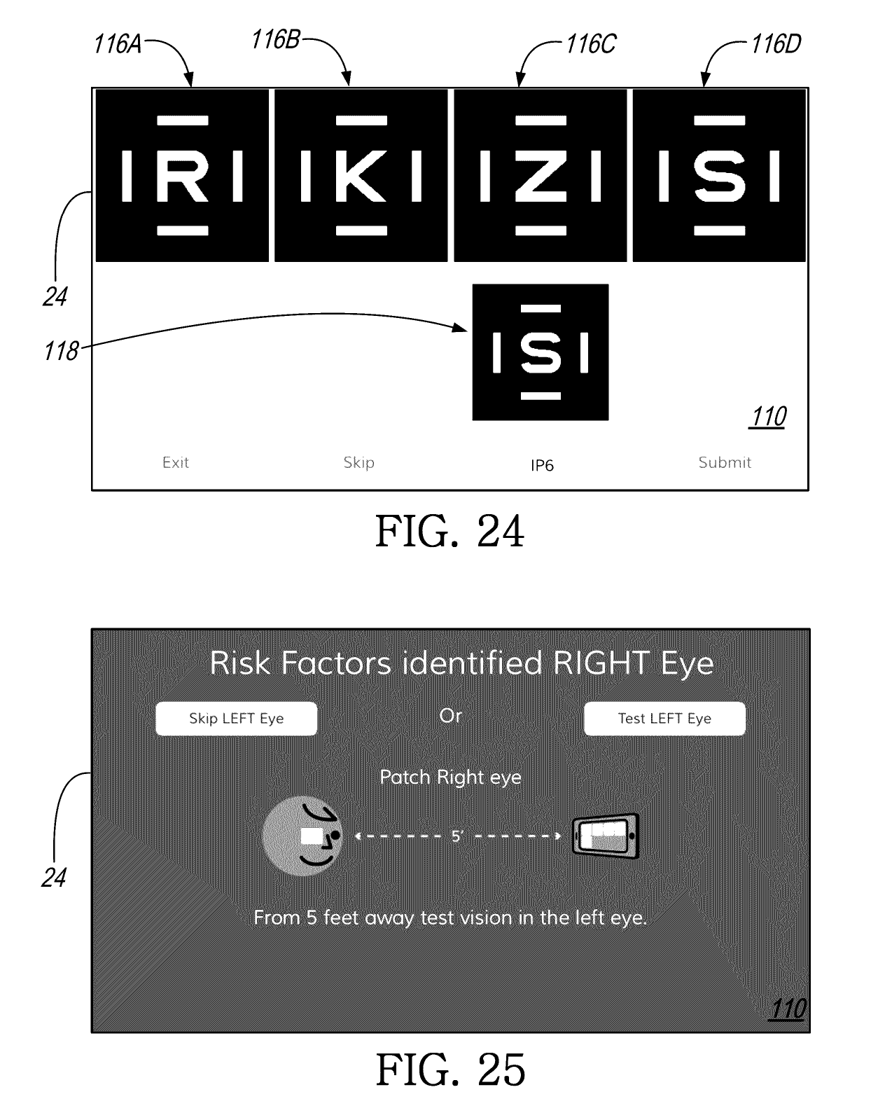

As the subject takes the test, the optotypes may get progressively smaller in successive rounds, corresponding to lines of distance visual acuity. For example, in a subsequent round after the first round, the application 50 may cause a smaller second visual acuity target 118 to be displayed along with the plurality of first visual acuity targets 116A-116D, as shown in FIG. 22. The second visual acuity target 118 displayed in a subsequent round may have a different optotype than in an initial round (see FIG. 23), and the order and/or positions of the plurality of first visual acuity targets 116A-116B may also be different in subsequent rounds (see FIG. 22). The size of the first visual acuity targets 116A-116D may be the same in the subsequent rounds, but displayed in a different order. In some embodiments, the size of the first visual acuity targets 116A-116D and/or the second visual acuity target 118 may change in subsequent rounds, as shown in FIG. 23. Those of ordinary skill in the art will appreciate that different visual acuity targets than shown in association with the visual acuity test 110 or the optotypes identified above, such as images of animals or cartoon characters, which help to maintain the interest of younger subjects. Other optotypes may be used other than HOTV, such as RKZS (see FIG. 24), ETDRS, or SLOAN, by way of non-limiting example. In some embodiments, the type of visual acuity targets used in one round may be different than the type of visual acuity target used in a different round. The Other arrangements of the visual acuity targets may be used, such as displaying the plurality of first visual acuity targets 116A-116D in a vertical line or concentrically arranged, and wherein the second visual acuity target 118 is movable along the direction of arrangement of the first visual acuity targets 116A-116D.

The test utilizes a clinically validated algorithm that presents different size optotypes, displayed multiple times, to determine whether the subject “passes” or “fails” a particular line of acuity. A fail is the inability to properly match the lower optotype with the upper optotype on at least two tries with a given size optotype. Once the acuity of one eye is ascertained, the user may be prompted to cover the eye already tested and test the other eye of the subject. The same procedure is applied to the eye until a final result is achieved. The test procedure may consider what optotype character size to test next based on the patient's result so far, the pattern of correct and wrong answers, the time delay for the patient to respond to each question, and the various stages of the test. The test procedure may attempt to minimize the number of questions in order to reduce the frustration of the patient.

In a critical line test procedure, comprehension is initially tested to determine whether the subject understands the test procedure process. If the subject passes comprehension, the assessment process advances to the critical line that is required to pass for a particular age. If the subject passes, the test is completed and the second eye may be tested in the same manner. If the subject fails the critical line, the critical line is tested a second time and if the subject fails again, the subject is identified with risk factors, as shown in FIG. 25. If risk factors are identified, the test may provide the option to advance the test to a different stage, such as testing the other eye of the subject.

In a near vision test procedure, the subject may be tested using a similar test to the threshold acuity test but with a closer test distance (for example, 14 inches from the user). The subject may also be tested with a paragraph style reading test at a close distance. The lines of text or letters get progressively smaller as the subject advances through the test.

Once the assessment is completed, the application 50 causes the computing device 12 to analyze the visual acuity information generated in step 210 (FIG. 13). The application 50 may determine a level of visual acuity for each eye of the subject. The level of visual acuity may correspond to one or more of distance vision for each eye, near vision for each eye, critical line vision, or binocular vision. The subject may be identified with risk factors based on age-specific referral criteria as defined by the American Academy of Pediatrics and the American Association of Pediatric Ophthalmology and Strabismus. The analysis may include calculating the visual acuity of each eye according to the threshold acuity testing (i.e., the ability to properly match the optotypes at a given size with a 1-20 feet distance D), determining whether the visual acuity “better than” or “worse than” the critical line displayed (if the critical line test was utilized), calculated near visual acuity (if the near vision test was utilized), and/or calculated binocular acuity (e.g., the best result from either eye).

After the visual acuity information is analyzed, the assessment process proceeds to step 212 (FIG. 13) where the results of the analysis are displayed on the display device 24 of the computing device 12, as shown in FIG. 8. The application 50 may display additional messages to the user about comprehension, time to administer the test, and provide additional descriptive results to the user. The application 50 may cause the computing device 12 to send visual acuity information generated and the results of the analysis to the server computing device 14, and/or store them on the data storage unit 34. The application may display an indication that risk factors have been identified as a result of the visual acuity information generated or the analysis performed, as shown in FIG. 25.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Likewise, any two components so associated can also be viewed as being “operably connected”, or “operably coupled”, to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

What is claimed is:

1. A handheld computing device for providing an ophthalmologic assessment for a subject's eyes, the computing device comprising:
an image-capturing device;
a display;
a data storage unit comprising an ophthalmologic health management application including programming data;
a processing unit operatively coupled to the image-capturing device, the display, and the data storage unit, execution of the programming data causing the processing unit to:
display, on the display, an interface of the ophthalmologic health management application on the display;
prompt a user to enter, via the interface, demographic information regarding the subject;
in response to receiving the demographic information, perform a demographic risk analysis based on the demographic information and generate recommendation information regarding one or more recommended ophthalmologic health assessments of a plurality of ophthalmologic health assessments recommended for performance based on the demographic risk analysis,
display the recommendation information on the display recommending performance of the one or more recommended ophthalmologic health assessments;
receive a selection of one of the plurality of ophthalmologic health assessments;
retrieve assessment information for performing the selected one of the plurality of ophthalmologic health assessments;
perform the selected one of the plurality of ophthalmologic health assessments to obtain ophthalmologic health information regarding at least one of the subject's eyes by controlling the image-capturing device and the display according to the assessment information;
perform an analysis on the ophthalmologic health information; and
display a result of the analysis on the display.

2. The computing device of claim 1, further comprising:
a light emitting device operatively coupled to the processing unit,
execution of the programming data further causing the processing unit to:
control the light emitting device to perform the selected one of the plurality of ophthalmologic health assessments to obtain the ophthalmologic health information regarding at least one of the subject's eyes by controlling the image-capturing device and the display according to the assessment information.

3. The computing device of claim 1, wherein execution of the programming data further causes the processing unit to:
retrieve second ophthalmologic assessment information of a previously performed ophthalmologic health assessment that resulted in positive diagnosis of an ophthalmologic condition, and diagnosis information regarding the previously performed ophthalmologic health assessment including an indicator of the positive diagnosis; and
display the second ophthalmologic assessment information in association with the diagnosis information on the display.

4. The computing device of claim 1, wherein execution of the programming data further causes the processing unit to:
transmit at least one of the ophthalmologic health information and the result to a database.

5. The computing device of claim 4, wherein execution of the programming data further causes the processing unit to:
prompt the user to input, via the interface, diagnosis information including whether the ophthalmologic health information presents symptoms of a positive diagnosis of an ophthalmologic condition; and
in response to receiving the diagnosis information from the user, transmit the diagnosis information to the database.

6. A computer-implemented method for assessing visual acuity of a subject, the method comprising:
displaying a first visual acuity target and a second visual acuity target on a display of a handheld computing device;
receiving a first measurement of an orientation of the handheld computing device about a first axis of the handheld computing device;
adjusting, according to the first measurement, a position of the second visual acuity target relative to a position of the first visual acuity target on the display;
receiving a selection specifying a current position of the second visual acuity target on the display as a selected position of the second visual acuity target;
responsive to receiving the selection, generating visual acuity information of the subject based at least in part on a determination regarding proximity of the selected position of the second visual acuity target relative to a position of the first visual acuity target;
analyzing the visual acuity information of the subject; and
displaying results of the analysis on the display.

7. The computer-implemented method of claim 6, further comprising:
simultaneously displaying a plurality of the first visual acuity targets on the display of the handheld computing device with the second visual acuity target, wherein
the position of the second visual acuity target is adjusted, according to the first measurement, relative to each of the plurality of first visual acuity targets on the display; and
the selected position of the second visual acuity target is analyzed relative to the plurality of first visual acuity targets, and the visual acuity information of the subject is generated based at least in part on whether a nearest one of the plurality of first visual acuity targets to the second visual acuity target matches the second visual acuity target.

8. The computer-implemented method of claim 7, wherein the plurality of first visual acuity targets comprise different optotypes and the second visual acuity target corresponds to one of the different optotypes of the plurality of first visual acuity targets, and the visual acuity information of the subject is generated based at least in part on whether the optotype of the second visual acuity target matches an optotype of the nearest one of the plurality of first visual acuity targets.

9. The computer-implemented method of claim 7, wherein the second visual acuity target has a different size than the plurality of first visual acuity targets.

10. The computer-implemented method of claim 7, wherein the plurality of first visual acuity targets are arranged along a first direction on the display, the position of the second visual acuity target is adjusted along a second direction on the display substantially parallel to the first direction.

11. The computer-implemented method of claim 7, further comprising:
displaying one or more other visual acuity targets on the display, wherein the visual acuity information includes information indicating the selected position of the second visual acuity target as a correct selection in response to a determination that the selected position of the second visual acuity target is nearer to the position of the first visual acuity target than to the one or more other visual acuity targets on the display.

12. The computer-implemented method of claim 11, wherein the first visual acuity target is a same type as the second visual acuity target, and the one or more other visual acuity targets are different types than the first visual acuity target and the second visual acuity target.

13. The computer-implemented method of claim 7, wherein the visual acuity information of the subject includes information indicating the selected position of the second visual acuity target as a correct selection in response to a determination that the selected position of the second visual acuity target is adjacent to the position of the first visual acuity target.

14. The computer-implemented method of claim 13, wherein
wherein the visual acuity information of the subject indicates a visual acuity level associated with the second visual acuity target, and the analysis results in a positive correlation between a visual acuity of the subject and the visual acuity level in response to the correct selection.

15. The computer-implemented method of claim 7, wherein the visual acuity information of the subject includes information indicating the selected position of the second visual acuity target as an incorrect selection in response to a determination that the selected position of the second visual acuity target is not adjacent to the position of the first visual acuity target.

16. The computer-implemented method of claim 6, further comprising:
receiving a second measurement of an orientation of the handheld computing device about a second axis orthogonal to the first axis, wherein
receiving the selection of the current position of the second visual acuity target on the display as the selected position is determined in response to detecting that the second measurement exceeds a first threshold.

17. The computer-implemented method of claim 6, further comprising:
displaying a third visual acuity target and a fourth visual acuity target on the display of the handheld computing device;
receiving a second measurement of the orientation of the handheld computing device about the first axis;
adjusting, according to the second measurement, a position of the fourth visual acuity target relative to a position of the third visual acuity target on the display of the handheld computing device; and
receiving a selection specifying a current position of the fourth visual acuity target on the display as a selected position of the fourth visual acuity target; and
responsive to receiving the selection specifying the selected position of the fourth visual acuity target, generating the visual acuity information of the subject based at least in part on a determination regarding proximity of the selected position of the fourth visual acuity target relative to a position of the third visual acuity target.

18. The computer-implemented method of claim 17, wherein the fourth visual acuity target is a different target than the second visual acuity target.

19. The computer-implemented method of claim 17, wherein the fourth visual acuity target is a different size than the second visual acuity target and the third visual acuity target.

20. The computer-implemented method of claim 17, wherein the third visual acuity target is a same size as the first visual acuity target.

21. The computer-implemented method of claim 17, wherein the fourth visual acuity target is a different optotype than the second visual acuity target.

22. The computer-implemented method of claim 6, further comprising:
displaying an indication prompting the subject to cover one of the subject's left eye and right eye, wherein the visual acuity information of the subject generated is associated with an other of the subject's left eye and right eye.

23. The computer-implemented method of claim 6, wherein the first visual acuity target and the second visual acuity target are displayed at a first time, and the method further comprises:
displaying, one or more additional times after the first time, responsive to receiving the selection specifying the selected position of the second visual acuity target, the first visual acuity target and the second visual acuity target;
receiving a second measurement of an orientation of the handheld computing device about the first axis;
adjusting, according to the second measurement, the position of the second visual acuity target relative to a position of the first visual acuity target on the display of the handheld computing device;
receiving a second selection specifying the current position of the second visual acuity target on the display as a second selected position of the second visual acuity target; and
responsive to receiving the second selection, generating the visual acuity information of the subject is based at least in part on a determination regarding proximity of the second selected position of the second visual acuity target relative to the position of the first visual acuity target.

24. The computer-implemented method of claim 6, the method further comprising:
capturing, using an image-capturing device of the handheld computing device, an image containing a left eye and a right eye of the subject;
analyzing the image captured to determine a distance between the image-capturing device and a face of the subject;
determining whether the distance is appropriate for performing a visual acuity assessment; and
in response to determining that the distance determined is inappropriate, providing an indication to adjust the distance.

25. A computer-implemented method for assessing visual acuity of a subject, the method comprising:
obtaining first visual acuity information regarding a first visual acuity level of the subject over one or more first test rounds, each test round comprising:
displaying a first visual acuity target and a second visual acuity target on a display of a handheld computing device;
receiving a first measurement of an orientation of the handheld computing device about a first axis;
receiving a first selection specifying a current position of the second visual acuity target as a selected position of the second visual acuity target; and
responsive to receiving the first selection, generating the first visual acuity information based at least in part on a determination regarding proximity of the selected position of the second visual acuity target relative to a position of the first visual acuity target; and
in response to obtaining the first visual acuity information over the one or more first test rounds,
analyzing the first visual acuity information of the subject; and
displaying results of the analysis of the first visual acuity information on the display.

26. The computer-implemented method of claim 25, wherein a size of the second visual acuity target is a same size for each of the one or more first test rounds.

27. The computer-implemented method of claim 25, further comprising:
obtaining second visual acuity information regarding a second visual acuity level of the subject over one or more second test rounds, each test round comprising:
displaying a third visual acuity target and a fourth visual acuity target on the display;
receiving a second measurement of an orientation of the handheld computing device about the first axis;
receiving a second selection specifying a current position of the fourth visual acuity target as a selected position of the fourth visual acuity target; and
responsive to receiving the second selection, generating the second visual acuity information based at least in part on a determination regarding proximity of the second selected position of the fourth visual acuity target relative to a position of the third visual acuity target; and
in response to obtaining the second visual acuity information over the one or more second test rounds,
analyzing the second visual acuity information of the subject; and
displaying results of the analysis of the second visual acuity information on the display.

28. The computer-implemented method of claim 27, wherein a size of the second visual acuity target is a same size for each of the one or more first test rounds, a size of the fourth visual acuity target is a same size for each of the one or more second test rounds, and the size of the fourth visual acuity target is different than the size of the second visual acuity target.

29. A handheld computing system for providing an ophthalmologic assessment for a subject's eyes, the handheld computing system comprising:
an image-capturing device;
a display;
an accelerometer;
a data storage unit comprising an ophthalmologic health assessment application including programming data;
a processing unit operatively coupled to the image-capturing device, the display, the accelerometer, and the data storage unit, execution of the programming data causing the processing unit to:
display, on the display, a first visual acuity target at a first position and a second visual acuity target at a second position;
receive, from the accelerometer, a first measurement of an orientation of the handheld computing device about a first axis;
adjust, according to the first measurement, a position of the second visual acuity target relative to a position of the first visual acuity target on the display;
receive a selection specifying an adjusted current position of the second visual acuity target on the display as a selected position of the second visual acuity target;
responsive to receiving the selection, generate visual acuity information of the subject based at least in part on a determination regarding proximity of the selected position of the second visual acuity target relative to a position of the first visual acuity target;
analyze the visual acuity information of the subject; and
display results of the analysis on the display.
